(19) United States
(12) Patent Application Publication
(10) Pub. No.: US 2001/0046966 A1
(43) Pub. Date: Nov. 29, 2001

(54) Inhibition of adipogenesis

(76) Inventors: Gokhan S. Hotamisligil, Cambridge, MA (US); Qiang Tong, Boston, MA (US)

Correspondence Address: MINTZ, LEVIN, COHN, FERRIS,, GLOVSKY and POPEO, P.C., One Financial Center, Boston, MA, 02111, US

(21) Appl. No.: 09/788,857

(22) Filed: Feb. 20, 2001

Related U.S. Application Data
(60) Provisional application No. 60/183,192, filed on Feb. 17, 2000.

(51) Int. Cl. A61K 48/00; A61K 38/17

(52) U.S. Cl. 514/44; 514/12

(57) ABSTRACT

The invention features compositions and methods for inhibiting adipogenesis by increasing GATA-2 or GATA-3 expression. Also included are methods of treating lipdystrophy by decreasing GATA-2 or GATA-3 expression.

This application claims priority to provisional patent application U.S. Ser. No. 60/183,192, filed Feb. 17, 2000.

### TECHNICAL FIELD

This invention relates to lipid metabolism disorders.

### BACKGROUND OF THE INVENTION

An estimated one-half of adults in the country are either overweight or obese. These conditions can least to a greater risk for developing a host of diseases, including diabetes, heart disease, stroke and certain cancers. Although a number of drugs or treatments for obesity are available, many have drawbacks such as undesirable side effects.

### SUMMARY

The invention is based on the discovery that transcription factors, GATA-2 and GATA-3, regulate fat cell formation. These transcription factors were detected in precursor cells that form fat cells. GATA-2 and GATA-3 regulate the extent to which precursor cells differentiate into mature adipocytes. In four different art recognized models of obesity, expression of GATA-2 and GATA-3 was decreased compared to the level of expression in wild type animals. GATA-2 and GATA-3 regulate the PPAR-gamma promoter, which is to critical for fat cell differentiation.

The invention features a method of blocking formation of adipocytes (adipogenesis) in a mammal by increasing GATA-2 or GATA-3 expression. For example, a nucleic acid encoding one or both transcription factor is administered to the mammal to aid in weight loss. Alternatively, a GATA-2 or GATA-3 gene product is administered. A compound which binds to a cis-acting regulatory region of GATA-2 or GATA-3 and upregulates transcription of these factors is used to increase intracellular GATA-2 or GATA-3 levels to therapeutically beneficial levels. Preferably, the therapeutic composition (nucleic acid or gene product) gains access to precursor fat cells to inhibit adipogenesis.

A method of inhibiting adipogenesis is carried out by contacting an adipocyte or adipocyte precursor with a compound that increases GATA-2 or GATA-3 expression. For example, the compound is a nucleic acid molecule encoding a GATA-2 polypeptide or an inducer of GATA-2 or GATA-3 expression. The compound binds to a cis-acting regulatory region of a GATA-2 or GATA-3 gene and augments transcription of a GATA-2 or GATA-3 gene.

A method of inhibiting adipogenesis is carried out by contacting an adipocyte or adipocyte precursor with a GATA-2 polypeptide. A method of inhibiting adipogenesis is also carried out by contacting an adipocyte or adipocyte precursor with a GATA-3 polypeptide. The polypeptide preferably contains a zinc finger domain. Preferably, the polypeptide contains at least 2 GATA zinc finger domains. The polypeptides and nucleic acids are isolated. They are separated from substances with which they naturally occur. For example, the polypeptides or nucleic acids are recombinant. Alternatively, they are purifed so as to achiev a purity of at least 75%. Preferably, the compound at leas 80%, 90%, 95%, and most preferably, 99-100% (by weight) the desired compound. Purity is evaluated using methods known in the art, e.g., electrophoretic and chromatographic techniques.

Obesity is treated by administering to a mammal a compound which increases expression of GATA-2 or GATA-3 or by administering to a mammal a GATA-2 polypeptide or a GATA-3 polypeptide.

A method of treating lipodystrophy in a mammal is carried out by administering to the mammal a compound which inhibits expression of GATA-2 or GATA-3. Lipid dystrophy (or lipdystrophy) is treated by modulating GATA expression, e.g., by inhibiting intracellular GATA production.

Other features, objects, and advantages of the invention will be apparent from the description and drawings.

### DESCRIPTION OF DRAWINGS

FIG. 1A is a photograph of an electrophoretic gel showing expression of GATA factors in adipose tissue. Total RNA was extracted from various tissues of male mice and the expression of GATA transcription factors were determined by northern blot analysis.

FIG. 1B is a photograph of an electrophoretic gel showing GATA expression in white (W) and brown (B) adipose tissues from three different strains of male mice. UCP1 is shown as a marker for brown adipose tissue and aP2 as a marker for both white and brown adipose tissues.

FIG. 1C is a photograph of an electrophoretic gel showing GATA expression in mouse white adipose tissue (WAT), mature adipocytes (AD), and stromal-vascular fraction (PRE). The expression of GATA transcription factors was determined by northern blot analysis and genes expressed in adipocytes (adipsin) and preadipocytes (AEBPI) are shown as controls. Ethidium Bromide (EtBr) staining was shown as a control for loading and integrity of total RNA.

FIG. 2 is a photograph of an electrophoretic gel showing GATA-2 and GATA-3 expression in cultured preadipocytes. 3T3-F442A cells were cultured to confluencey (day 0) and incubated with 5 ug/ml insulin to stimulate differentiation into adipocytes. Plates of cells collected for RNA isolation before the induction of differentiation and with 2-day intervals thereafter. Total RNA was isolated from these cells using the TRIZOL reagent (GIBCO) and the expression of GATA transcription factor and differentiation-dependent adipocyte markers (PPAR and adipsin) were determined by northern blot analysis. Ethidium Bromide (EtBr) staining was shown as a control for loading and integrity of total RNA.

FIG. 3A is a photograph of Oil Red-O stained cells showing the effect of constitutive GATA-2 and GATA-3 expression on adipocyte differentiation. Constitutive expression of GATA transcription factors in 3T3 -F442A cells was achieved by using a retro-viral expression system. These cells were treated with 5 ug/ml insulin to stimulate differentiation. The extent of differentiation was determined by Oil Red-O staining which reveals lipid accumulation.

FIG. 3B is a photograph of an electrophoretic gel showing expression of differentiation-dependent adipogenic genes (aP2, Glut4, adipsin, and PPAR-gamma) and genes expressed in preadipocytes (Pref-1 and AEBP-1). Ethidium Bromide (EtBr) staining was shown as a control for loading and integrity of total RNA. PreAd, vector infected 3T3-F442A cells without induction of differentiation. Lanes labeled Ad, GATA-2, GATA-3 are vector, GATA-2 and GATA-3 expressing 3T3-F442A cells, respectively, induced to differentiate for six days.

FIG. 4A is a bar graph showing the effect of GATA-2 and GATA-3 on PPAR-gamma2. A PPAR-gamma2 proximal promoter fragment (nucleotide −603 to +62) was amplified by PCR and inserted into the pXP2 plasmid. The resulting luciferase reporter construct (lug) was transected into NIH3T3 cells with 5 ug of PCDNA-GATA-2 (A) or Pcdna-gata-3 expression plasmid.

FIG. 4B is a bar graph showing the effect of GATA-2 and GATA-3 on PPAR-gamma2. Calcium phosphate method was used to introduce DNA into the test cells. 0.5 ug of a Renilla luciferase reporter construct (pRL-TK, Promega) was also included in the mixture, serving as a control of transfection efficiency. Two days after transfection, both firefly and Renilla luciferase activities were determined in 20 ul of total cells lysate, using Dual Luciferase Assay System from Promega. The PPAR-gamma 2 promoter activity was normalized with the transfected DNA and the value was set to 1 for vector control. The data shown are the average of three experiments.

FIG. 5A is a photograph of an electrophoretic gel of a DNA footprinting analysis which identified GATA-2 and GATA-3 binding sites on PPAR-gamma 2 promoter. A ³²P-end labeled DNA fragment (nucleotide −264 to +62) was mixed with or without 5 ul of recombinant GST-GATA-2 fusion in a total volume of 50 ul. After 20 min. incubation at room temperature, 3 ul of 1:20 diluted RQ1 Dnase (Promega) was added for 1 min.

FIG. 5B is a photograph of an electrophoretic gel showing the results of an mobility shift assay. Based on the result of Dnase I footprinting, two double stranded DNA fragment (nucleotide −199 to −85 and nucleotide −22 to +9, respectively) were end-labeled with 32P and electrophoretic mobility shift assays were performed with GST-GATA-2 or GST-GATA-3. For each protein-DNA reaction, specific (S) or non-specific (N) DNA competitors were also included in the experiments.

FIG. 5C is a bar graph showing GATA responsiveness of 370 bp (nucleotide −361 to +9) PPAR-gamma 2 promoter DNA fragment is mediated by two GATA binding sites. The 370 bp PPAR- gamma 2 promoter contains the two GATA binding sites shown in FIGS. 5A and B. Introduction of mutations into these two GATA binding sites (GGAT to CCTT) at nucleotide 112 to −108 and AATC to AAAT at nucleotide −1 to +3) alleviated the suppressive effect of GATA on the PPAR-gamma 2 promoter.

FIG. 6 is a photograph of an electrophoretic gel showing severely defective adipose expression of GATA-2 and GATA-3 in murine models of obesity. White adipose tissue samples were obtained from four different genetic models of murine obesity and GATA expression was examined by northern blot analysis. The db/db, KKA yellow and tub/tub mice along with the appropriate controls were obtained from Jackson Laboratories (Bar Harbor, Maine). The ob/ob mice and the controls were from our own colony. Genes with decreased (adipsin), increased (leptin) and unchanged (aP2) expression levels between the adipose tissues of lean and obese animals are shown as controls. L: lean, 0: obese, Ethidiumn Bromide (EtBr) staining was shown as a control for loading and integrity of total RNA.

### DETAILED DESCRIPTION

Adipocyte differentiation is a multistep process controlled by the action of a complex interactive network of transcription factors in mammals. In the fruit fly the serpent gene is critical for the genesis of the fruit fly fat body, which corresponds to mammalian liver and adipose tissue. The function of the GATA family of transcription factors in adipogenesis in mice was investigated since they are the mammalian homotogues of Drosophila serpent gene. Highly specific expression of GATA-2 and GATA-3 was demonstrated in adult mice adipose tissues. GATA-3 expression was restricted to only adipocyte precursors in white but not brown adipose tissue, establishing the first known gene expressed in this fashion. In cultured cells, GATA-2 and GATA-3 expression was detected only in preadipocytes and dramatically downregulated upon differentiation. Constitutive expression of both GATA-2 and GATA-3 suppressed adipocyte differentiation and trapped the cells at the preadipocyte stage. Both GATA-2 and GATA-3 directly bind to specific sites in the proximal promoter of the adipogenic transcription factor, peroxisome proliferaror activated receptor-gamma (PPAR-gamma), and negatively regulate its activity. Finally, both GATA-2 and. GATA-3 expression is severely defective in the white adipose tissue of several different models of obesity. These results indicate that GATA-2 and GATA-3 are preadipocyte markers and play an important role in adipogenesis.

Proper development and maintenance of adipose tissue is critical for the survival of the vertebrate organism. Besides its role in energy storage and release, adipose tissue is also an active player in endocrine homeostasis at multiple sites in the body There are two general classes of fat cells in mammals, brown and white. The classic hallmark of white adipose tissue (WAT) is its ability to store excess energy in the form of triglyceride and release free fatty acids during caloric deficiency. Brown adipose tissue (BAT), on the other hand, is characterized by its ability to dissipate energy through thermogenesis.

The coordinated action of PPAR-gamma and CCAAT/enhancer binding protein (C/EBP) family of transcription factors plays a central role in the regulation of the adipocyte differentiation program. Subsequent to C/EBP beta and C/EBP sigma expression during differentiation of adipocytes. C/EBPalpha and PPAR-gamma production is stimulated. There is a positive feedback loop between PPAR-gamma and C/EBP alpha, both factors induce the expression of the other. This powerful synergy between PPAR-gamma and the members of the C/EBP family drives the expression of a multitude of genes that are necessary for the generation and maintenance of the adipogenic phenotype. Genetic absence of these factors severely limits the capacity of cells to form adipocytes.

Although considerable progress has been made to elucidate the molecular mechanisms of adipogenesis, little is known about the earlier stages of differentiation, the commitment of pluripotent stem cells into adipogeneric lineages and genes that control the transition from preadipocytes to adipocytes. To identify such factors, studies were undertaken to determine whether the genes that are critical in the formation of the Drosophila melanogaster fat body are conserved in mammals. Fat body is the Drosophila homologue of mammalian adipose tissue and liver. Several genes have been reported to be expressed specifically in this structure and potentially important in its development.

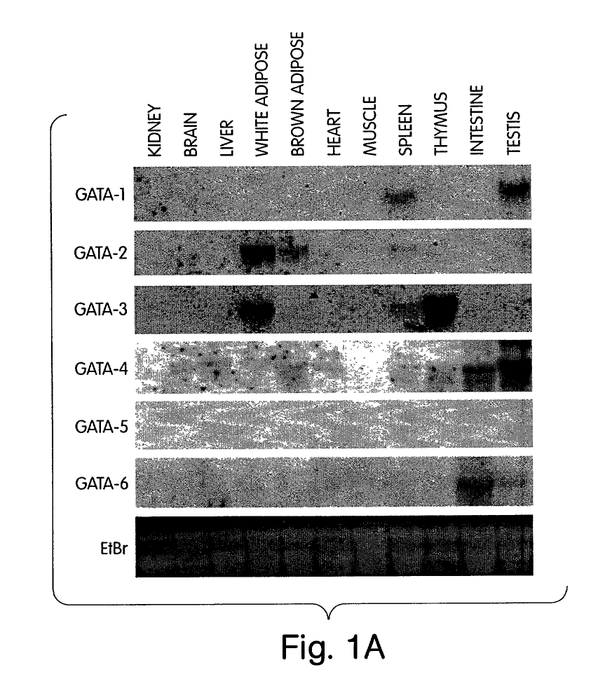

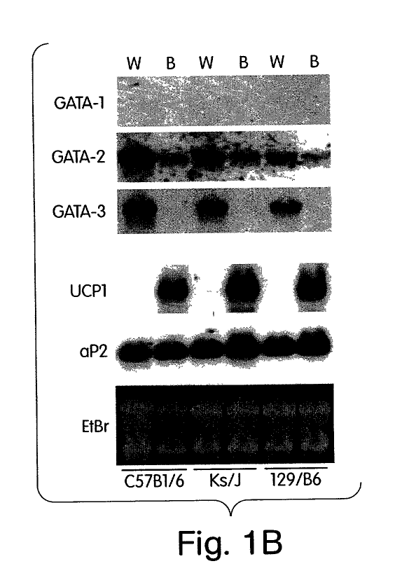

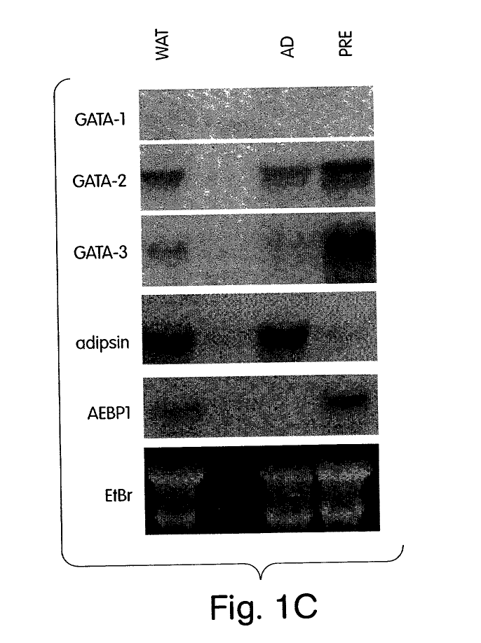

Drosophila serpent (srp) gene is a member of the GATA transcription factor family which shares a highly conserved zinc-finger DNA binding domain and binds specifically to a consensus DNA sequence (A/T)GATA(A/G) (SEQ ID NO:1). It has been demonstrated as a critical gene for the formation of fly fat body. Expression of mammalian GATA factors was measured in mouse adipose tissue by Northern blot analysis. Among the six GATA factors investigated, GATA-2 and GATA-3 expression was evident in white adipose tissue (FIG. 1A). Strikingly, only GATA-2, but not GATA-3 mRNA was also detected in brown adipose tissue. GATA-1, GATA-4, GATA-5 and GATA-6 expression was not observed in either fat depot by northern blot analysis. Screening of adipocyte-derived libraries also did not reveal any novel members at this site. Since the white adipose tissue-restricted expression pattern observed for GATA-3 has not been noted for any other gene, GATA-3 expression was examined in additional strains of mice to rule out effects related to a specific genetic background. As shown in FIG. 1B, GATA-3 gene is expressed only in white but not in brown adipose tissue in several independent strains of mice.

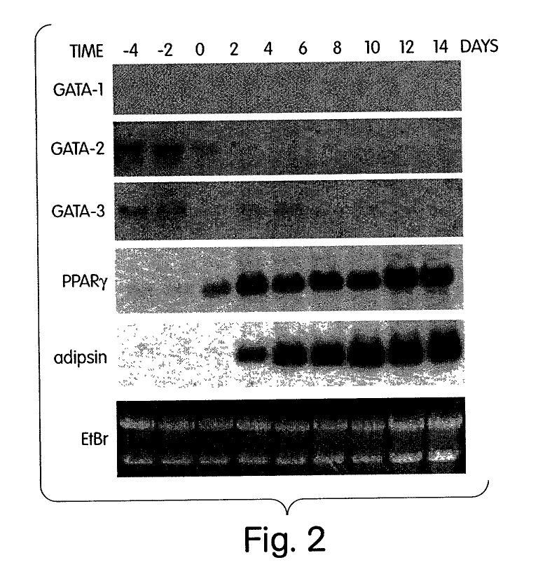

Adipose tissue contains mature adipocytes, adipocyte precursors and other cell types such as vascular endothelial or smooth muscle cells and macrophages. To determine the source of GATA expression, adipose tissue was fractionated to examine GATA-2 and GATA-3 expression in mature adipocytes arid the stromal-vascular fraction. As shown in FIG. 1C, both GATA-2 and GATA-3 are expressed preferentially in the stromal-vascular fraction, which contains adipocyte precursors. Taken together with the results shown in FIG. 1A, the source of GATA-2 and GATA-3 is the preadipocytes since the expression of these genes was not observed in other tissues that are rich in other cell types (FIG. 1A). In fact, GATA-2 expression in white fat is the highest among all of the adult tissues investigated and GATA-3 expression in white fat is second only to thymus. The expression and regulation of GATA-2 and GATA-3 was measured in pure populations of cultured 3T3-F442A preadipocytes. As shown in FIG. 2, GATA-2 and GATA-3 mRNA expression was detected in 3T3-F442A preadipocyes. Their expression was dramatically down-regulated when these preadipocytes were differentiated into adipocytes (FIG. 2). Identical results were also obtained in the 3T3-L1 preadipocyte cell line demonstrating that, as seen in the primary isolated mature adipocytes. GATA-2 or GATA-3 expression was only detectable in precursors but not in fully differentiated clonal adipocytes in culture.

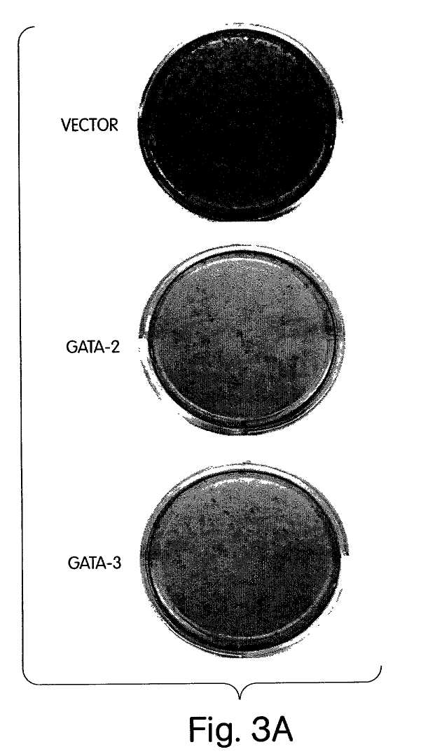

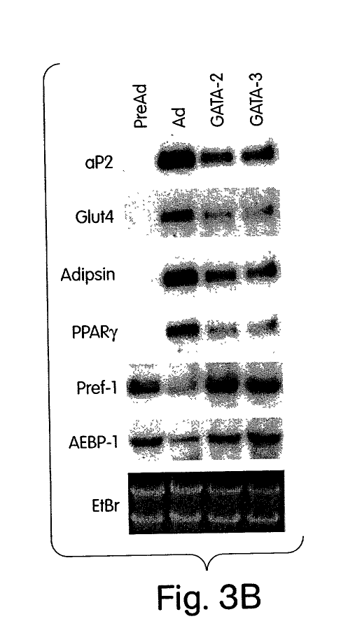

The data indicated that GATA-2 and GATA-3 are potential preadipocyte markers and are likely play an important role in preadipocyte commitment and/or regulation of adipocyte differentiation. A retroviral expression system was developed to constitutively express individual GATA factors in 3T3-F442A preadipocytes. This system allowed investigation of a large number of cells and overcame the limitations of individual clones. The expression of each GATA factor was confirmed by northern blot analysis. 3T3-F442A preadipocytes constitutively expressing GATA-2 and GATA-3 were tested for their ability to differentiate into adipocytes. The extent of differentiation was evaluated by microscopy, Oil Red O staining of intracellular lipid droplets and examination of molecular markers of adipogenesis. Microscopic examination and severely reduced neutral lipid accumulation (FIG. 3A) demonstrate that unregulated expression of GATA-2 and GATA-3 resulted in strong inhibition of adipocyte differentiation in 3T3-F442A cells compared to those expressing the retroviral vector alone.

To further illustrate the extent of differentiation of these cells, a panel of markers of adipocyte differentiation, such as PPAR-gamma, Glut4, aP2, and adipsin was examined (FIG. 3B). Six days after the initiation of differentiation, cells infected with the viral vector alone demonstrated extensive conversion into adipocytes (FIG. 3A) with elevated expression of PPAR-gamma, C/EBP alpha, Glut4, aP2, and adipsin (FIG. 3B, lane 2), compared to undifferentiated controls (FIG. 3B, lane 1). In contrast, cells expressing GATA factors differentiation was suppressed and the mRNA levels of all of these differentiation-dependent genes were significantly reduced. Previous studies have reported the identification of several genes such as Pref-1, AEBP-1, and AP-2 alpha 1 that are expressed in preadipocytes and downregulated in mature adipocytes (FIG. 3B). In line with these findings, the expression of both Pref-1 and AEBP-1 were found to be down-regulated upon differentiation in control cells expressing vector alone. However, in GATA-expressing cells, expression of both Pref-1 and AEBP-1 is maintained at a level comparable to undifferentiated preadipocytes. Taken together, these results demonstrate that GATA-expressing cells were trapped at the preadiocyte stage. These experiments were conducted with pools of thousands of clones of retrovirally infected cells with variable levels of expression. The fact that constitutive expression of GATA-2 and GATA-3 is not permissive for adipogenesis in this system reflects the robustness of GATA function during adipocyte differentiation.

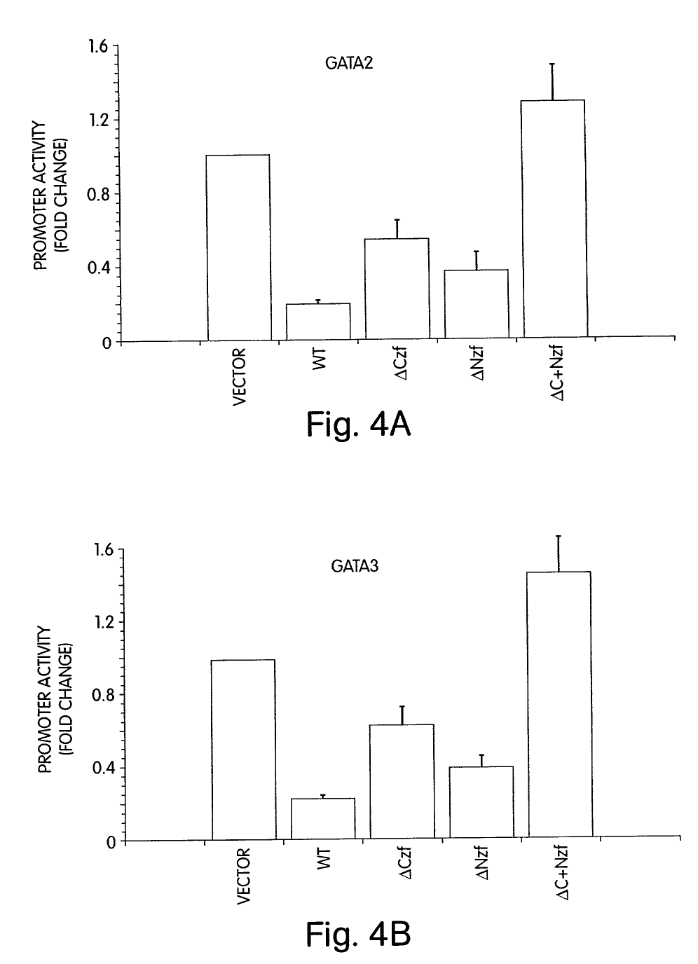

The potential molecular mechanisms by which GATA factors regulate adipocytes differentiation was investigated. Based on the central role of PPAR-gamma in adipogenesis, the possibility that GATA-2 and GATA-3 may directly regulate the activity of this transcription factor was studied. Examination of the 0.6 kb proximal PPAR-gamma 2 promoter region revealed several potential GATA binding sites. Studies were then undertaken to test the ability of GATA-2 or GATA-3 to directly regulate the activity of PPAR-gamma 2 promoter. The activity of a luciferase reporter gene driven by the 0.6 kb (nucleotides −603 to +62) PPAR-gamma 2 promoter in NIH3T3 cells in the presence and absence of GATA-2 or GATA-3. As shown in FIGS. 4A-B, both GATA-2 and GATA-3 significantly suppressed (5-fold) the activity of the 0.6 kb PPAR promoter. In the same experimental setting, estrogen receptor response element-driven luciferase activity was enhanced by GATA-2 indicating the specificity of the GATA-mediated suppression of PPAR-gamma 2 promoter. While GATA factors are transcription factors with DNA binding capacity, it is necessary to determine whether they exert this particular function through direct DNA binding and transcriptional regulation. It has been demonstrated that the carboxyl zinc fingers of GATA-2 and GATA-3 are responsible for DNA binding, and the amino zinc finger is implicated in stabilizing DNA binding as well as protein-protein interactions.

The cDNA expression clones with deletions in one or both zinc fingers of GATA-2 or GATA-3 were generated by litigation of two PCR amplified fragments flanking the targeted region. The GATA-2 carboxyl zinc finger deletion mutant lacks amino acid residues 342 to 367. The GATA-2 amino zinc finger deletion mutant tacks amino acid residues 281-313. The deletion of both zinc fingers of GATA-2 lacks amino acid residues 281 to 367. The GATA-3 carboxyl zinc finger deletion mutant lacks amino acid residues 317-341. The GATA-3 amino zinc finger deletion mutant lacks amino acid residues 257-287. The deletion of both zinc fingers of GATA-2 lacks amino acid residues 257 to 341. The recombinant GATA-2 was expressed as a fusion protein with glutathione S-transferase (GST) in the pGEX-2T plasmid (Pharmacia). Following induction expression with 0.3 mM IPTG for two hours, the fusion protein is affinity purified with glutathione Sepharose (SIGMA).

As shown in FIGS. 4A-B, the deletion of both zinc finger domains completely reversed the suppression of PPAR-gamma 2 promoter activity by both GATA factors. Deletion of the carboxyl zinc finger of either GATA-2 or GATA-3 also significantly but incompletely reversed their ability to suppress PPAR-gamma 2 promoter. A similar but smaller effect was observed upon deletion of the of amino zinc finger. All three GATA mutants were expressed at comparable levels to wild type protein. These data demonstrate that while the carboxyl zinc finger of GATA-2 and GATA-3 is dominant, both zinc fingers are necessary for GATA factors to fully interact with the PPAR-gamma 2 promoter and suppress its activity.

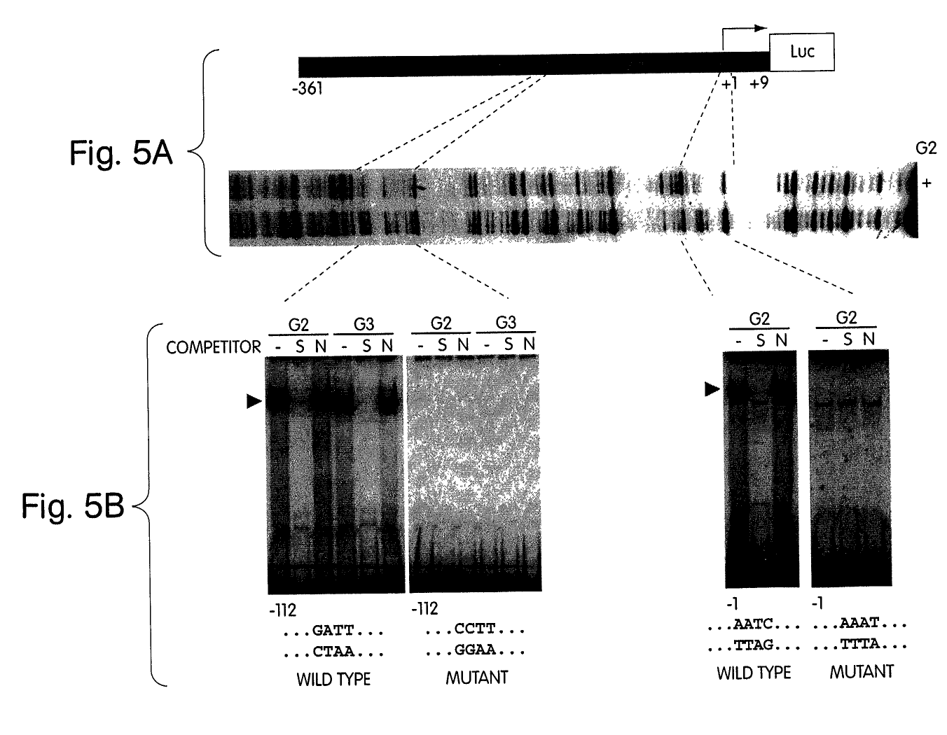

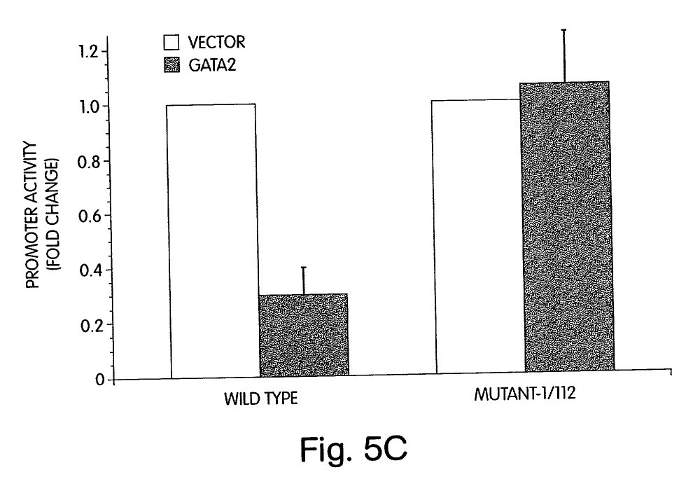

After confirming the requirement for GATA DNA-binding activity, experiments were carried out to determine whether this activity requires direct interaction with the PPAR-gamma 2 promoter. The proximal PPAR-gamma 2 promoter region was evaluated by deletion analysis. These studies demonstrated that a 370 bp fragment (nucleotide −361 to +9) still retains basal promoter activity and suppressed by GATA. To determine if and at what sites GATA factors directly interact with DNA on this fragment, DNase I footprinting experiments were carried out (FIG. 5A) combined with elecrophoretic mobility shift assays (FIG. 5B) using recombinant GATA-2 and GATA-3 fusion proteins. The electrophoretic mobility shift assays were performed in 20 ul reaction volume. 2 ul GST-GATA fusion protein was incubated with 0.5 ug poly[d(I-C)] and ³²p labeled DNA probe (4×10⁴ cpm) in the presence or absence of 1 ug of specific or non-specific competitors, at room temperature for 20 min. The sequence of the specific competitor was 5′-GATCTCCGGCAACTGATAAGGATTCCCTG-3′ (SEQ ID NO:2) and the sequence of the non-specific competitor was 5′-GATCGAACTGACCGCCCGCGGCCCGT-3′ (SEQ ID NO:3).

DNase I footprinting revealed two consistent GATA binding sites at positions −112 and −1. Sequence-dependent GATA binding to these sites was further demonstrated by the formation of specific DNA-protein complexes in electropborctic mobility shift assays (FIG. 5B). The formation of GATA protein-DNA complexes was competed only by a specific DNA fragment with known GATA binding site and supershifted by specific anti-GATA antibodies confirming specificity (FIG. 5B). Glutathione S-transferase (GST) alone had no capacity to interact with the DNA targets used in these experiments.

Point mutations were introduced into these two GATA-binding sites within the 370 bp promoter region. These mutations completely abolished GATA binding as demonstrated by mobility shift assays (FIG. 5B). To test whether suppression of the PPAR-gamma promoter is dependent on direct GATA interaction at these specific sites, a reporter gene was expressed driven by the 370 bp PPAR-gamma 2 promoter harboring mutations in GATA binding sites at positions −112 and −1. GATA binding sites span a region of at least 10 nucleotides containing nucleotide −112 or a region of at least 10 nucleotides containing nucleotide −1. FIG. 5C shows that the activity of the 370 bp fragment of PPAR-gamma 2 lacking both GATA-binding Sites was not suppressed by GATA factors. Introduction of mutations into either site, alone, was insufficient to prevent GATA-mediated suppression. Taken together, the results demonstrate that GATA-2 and GATA-3 act directly on two specific sites on the proximal PPAR-gamma promoter and negatively regulate its transcriptional activity.

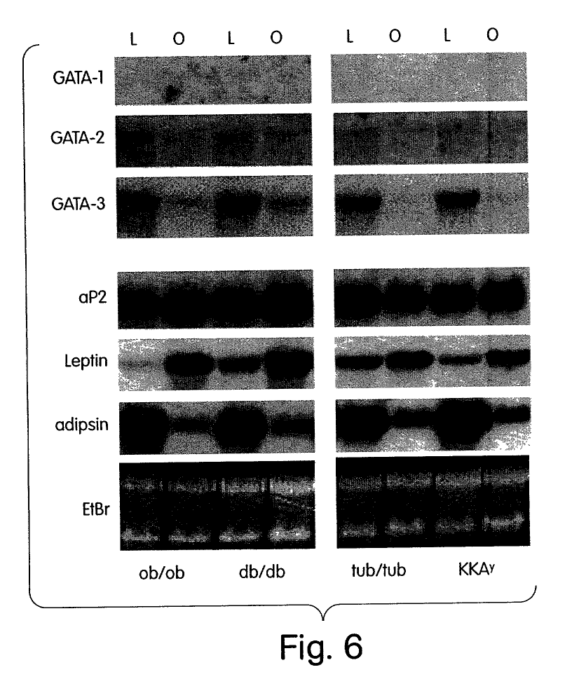

GATA-2 and GATA-3 regulate the transition from preadipocytes to adipocytes and defects in the expression and/or function of such genes are associated with increased adiposity. The expression of GATA-2 and GATA-3 was measured in white adipose tissues of several different models of murine obesity. These experiments demonstrated a severe deficiency in the adipose expression of both GATA-2 and GATA-3 in four independent genetic models of obesity including ob/ob, db/db, tub/tub, and KKA yellow compared to matched lean littermates (FIG. 6). These data indicate that obesity is associated with a dramatic loss of GATA-2 and GATA-3 expression in adipose tissue independent of its etiology.

Taken together our data indicate that GATA-2 and GATA-3 are preadipocyte genes, which act as molecular gatekeepers by controlling the transition from preadipocytes to adipocytes. In higher organisms with balanced energy homeostasis, only a portion of the preadipocyte pool is utilized to become differentiated adipocytes. The rest will remain quiescent. Under the appropriate conditions such as imbalance between energy intake and output, these cells differentiate into adipocytes and expand adiposity. Similar regulation would be necessary for brown adipose tissue in times of high demand for thermogenesis. If these control points fail, the result will be increased adiposity and consequently a higher tendency for obesity. The cellular machinery is equipped with molecules to control the rate and extent of transition between preadipocytes and adipocytes. The data presented here indicates that GATA-2 and GATA-3 play a role in this control mechanism.

The data also demonstrate that at least one potential mechanistic basis for this GATA action is direct negative regulation of the PPAR-gamma 2 promoter activity. In most experimental systems, GATA transcription factors act as transcriptional activators. Their function as a transcriptional suppressor is unusual, although, not unprecedented. Hence, the strong biology associated with the GATA-mediated negative regulation of PPAR-gamma promoter activity provides a useful experimental paradigm to further investigate the cellular and molecular requirements for this action of GATA factors and regulation of PPAR activity in a broader spectrum.

Unlike-GATA-2, GATA-3 is expressed only in white but not in the brown adipose tissue. No other known gene is expressed in this fashion. Understanding the molecular basis of this highly unusual expression pattern should yield novel insights into the control of gene expression in these lineages. It is generally assumed that during the formation of adipocytes, pluripotent stem cells with mesodermal origin commit to the preadipocyte lineage and further terminally differentiate into mature adipocytes under permissive conditions. Cell lineage determination and terminal differentiation is achieved, in part, at the transcription level through tissue-specific transcription factors and the activation of expression of tissue-specific genes. Since both GATA-2 and GATA-3 expression is restricted to adipocyte precursors and mutation of the serpent gene in Drosophila is characterized by failure to develop the fat body, the coordinated activity of GATA factors might also be critical in the commitment to preadipogenic lineages and formation of adipose tissue. However, total absence of both GATA-2 and GATA-3 results in early embryonic lethality in mice.

The importance of GATA function in adipose tissue has been preserved from the fruit fly to the mouse, although the architecture and the molecular complexity of the tissue have evolved significantly. This high degree of evolutionary conservation indicates a fundamental role for GATA in the biology of adipose tissue.

Therapeutic Administration

Mammals such a humans which are overweight, obese, or at risk of becoming so are treated with compounds which increase GATA-2 or GATA-3 expression or activity. By “activity” is meant binding to a site in the proximal promoter of the PPAR-gamma gene. Binding negatively regulates promoter activity. Method of determining whether or not an individual is overweight or obese are known in the art. For example, Body mass index (BMI) is measured (kg/m² (or lb/in²×704.5)). Alternatively, waist circumference (estimates fat distribution), waist-to-hip ratio (estimates fat distribution), skinfold thickness (if measured at several sites, estimates fat distribution), or bioimpedance (based on principle that lean mass conducts current better than fat mass (i.e. fat mass impedes current), estimates % fat) is measured. The parameters for normal, overweight, or obese individuals is as follows: Underweight: BMI<18.5; Normal: BMI 18.5 to 24.9; Overweight: BMI=25 to 29.9. Overweight individuals are characterized as having a waist circumferenceof >94 cm for men or >80 cm for women and waist to hip ratios of ≧0.95 in men and ≧0.80 in women. Obese individuals are characterized as having a BMI of 30 to 34.9, being greater than 20% above “normal” weight for height, having a body fat percentage >30% for women and 25% for men, and having a waist circumference >102 cm (40 inches) for men or 88 cm (35 inches) for women. Individuals with severe or morbid obesity are characterized as having a BMI of ≧35.

GATA-2 or GATA-2 is therapeutically overexpressed (e.g., by administering an inducing agent) to increase expression from the endogenous gene or by administering DNA (alone or in a plasmid) encoding an GATA gene product under the control of a strong inducible or constitutive promoter. Human GATA-2 amino acid sequences are known in the art (see, e.g., Lee et al., 1991, J. Biol. Chem. 266:16188-16192; GENBANK™ Accession Nos.P23769, A41782, A40815, or AAA35869). Nucleic acid sequences encoding human GATA-2 are also known in the art (e.g., GENANK™ Accession Nos. M77810 or M68891). Human GATA-3 is described in Ho et al., 1991, EMBO J 10:1187-1192, the amino acid sequence is described in GENBANK™ Accession No. CAA28877, and the nucleic acid encoding human GATA-3 is defined at GENBANK™ Accession No. AR106369, X55122, or X73519).

Inducers of GATA-2 or GATA-3 expression are identified by incubating a promoter region operably linked to a reporter sequence with a candidate compound. An increase in transcription of the reporter gene (or an increase in the amount of the reporter gene product) in the presence of the candidate compound compared to the level in the absence of the compound indicates that the compound increases GATA-2 or GATA-3 expression. Such promoter sequences are known in the art. A decrease in the level of expression of the reporter gene or gene product in the presence of the candidate compound compared to the level in the absence of the compound indicates that the compound inhibits GATA-2 or GATA-3 expression (e.g., intracellular GATA production). For example, the human GATA-2 promoter region is available at GENBANK™ Accession No. U79137. GATA-3 regulatory regions are available at GENBANK™ Accession No.AJ131811.

For local administration of DNA, standard gene therapy vectors used. Such vectors include viral vectors, including those derived from replication-defective hepatitis viruses (e.g., HBV and HCV), retroviruses (see, e.g., WO 89/07136; Rosenberg et al., 1990, N. Eng. J. Med. 323(9):570-578), adenovirus (see, e.g., Morsey et al., 1993, J. Cell. Biochem., Supp. 17E,), adeno-associated virus (Kotin et al., 1990, Proc. Natl. Acad. Sci. USA 87:2211-2215,), replication defective herpes simplex viruses (HSV; Lu et al., 1992, Abstract, page 66, Abstracts of the Meeting on Gene Therapy, September 22-26, Cold Spring Harbor Laboratory, Cold Spring Harbor, N.Y.), and any modified versions of these vectors. The invention may utilize any other delivery system which accomplishes in vivo transfer of nucleic acids into eucaryotic cells. For example, the nucleic acids may be packaged into liposomes, e.g., cationic liposomes (Lipofectin), receptor-mediated delivery systems, non-viral nucleic acid-based vectors, erytbrocyte ghosts, or microspheres (e.g., microparticles; see, e.g., U.S. Pat. No. 4,789,734; U.S. Pat. No. 4,925,673; U.S. Pat. No. 3,625,214; Gregoriadis, 1979, Drug Carriers in Biology and Medicine, pp. 287-341 (Academic Press,). Naked DNA may also be administered.

DNA for gene therapy can be administered to patients parenterally, e.g., intravenously, subcutaneously, intramuscularly, and intraperitoneally. DNA or an inducing agent is administered in a pharmaceutically acceptable carrier, i.e., a biologically compatible vehicle which is suitable for administration to an animal e.g., physiological saline. A therapeutically effective amount is an amount which is capable of producing a medically desirable result, e.g., expression or overexpression of a GATA gene product in a treated animal. Such an amount can be determined by one of ordinary skill in the art. As is well known in the medical arts, dosage for any given patient depends upon many factors, including the patient's size, body surface area, age, the particular compound to be administered, sex, time and route of administration, general health, severity of arteriosclerosis or vascular injury, and other drugs being administered concurrently. Dosages will vary, but a preferred dosage for intravenous administration of DNA is approximately 10⁶ to 10²² copies of the DNA molecule.

GATA gene products are administered to the patient intravenously in a pharmaceutically acceptable carrier such as physiological saline. Standard methods for intracellular delivery of peptides can be used, e.g. packaged in liposomes. Such methods are well known to those of ordinary skill in the art. It is expected that an intravenous dosage of approximately 1 to 100 moles of the polypeptide of the invention would be administered per kg of body weight per day. The compositions of the invention are useful for parenteral administration, such as intravenous, subcutaneous, intramuscular, and intraperitoneal.

Compounds which inhibit or decrease GATA-2 or GATA-3 expression or intracellular polypeptide production are useful to treat adipose tissue deficiencies or lipid dystrophy (lipodystrophy). Methods of identifying mammals suffering from or at risk of developing such conditions are known in the art.

Other embodiments are within the following claims.

What is claimed is:

1. A method of inhibiting adipogenesis, comprising contacting an adipocyte or adipocyte precursor with a compound that increases GATA-2 or GATA-3 expression.

2. The method of claim 1, wherein said compound increases GATA-2 expression.

3. The method of claim 1, wherein said compound increases GATA-3 expression.

4. The method of claim 1, wherein said compound is a nucleic acid molecule encoding a GATA-2 polypeptide.

5. The method of claim 1, wherein said compound is an inducer of GATA-2 expression.

6. The method of claim 1, wherein said compound is an inducer of GATA-3 expression.

7. The method of claim 5 or 6, wherein said compound binds to a cis-acting regulatory region of a GATA-2 or GATA-3 gene and augments transcription of a GATA-2 or GATA-3 gene.

8. A method of inhibiting adipogenesis, comprising contacting an adipocyte or adipocyte precursor with a GATA-2 polypeptide.

9. A method of inhibiting adipogenesis, comprising contacting an adipocyte or adipocyte precursor with a GATA-3 polypeptide.

10. The method of claim 8 or 9, wherein said polypeptide comprises a zinc finger domain.

11. A method of treating obesity, comprising administering to a mammal a compound which increases expression of GATA-2 or GATA-3.

12. A method of treating obesity, comprising administering to a mammal a polypeptide selected from the group consisting of a GATA-2 polypeptide or a GATA-3 polypeptide.

13. The method of claim 12, wherein said polypeptide is a GATA-2 polypeptide.

14. The method of claim 12, wherein said polypeptide is a GATA-3 polypeptide.

15. A method of treating lipodystrophy in a mammal comprising administering to said mammal a compound which inhibits expression of GATA-2 or GATA-3.

16. The method of claim 15, wherein said compound inhibits expression of GATA-2.

17. The method of claim 15, wherein said compound inhibits expression of GATA-3.
